(19) United States
(12) Patent Application Publication
(10) Pub. No.: US 2026/0013983 A1
(43) Pub. Date: Jan. 15, 2026

(54) INTRAOCULAR LENSES AND METHODS OF ADJUSTING A CYLINDER POWER OF AN INTRAOCULAR LENS

(71) Applicants: Terah Whiting SMILEY, Davis, CA (US); Venkata Mohan KUDAPA, Foster City, CA (US); Joselito SORIANO, Hayward, CA (US); Nathan LEWIS, San Jose, CA (US); Khoi VU, San Jose, CA (US); Matthew Joseph LANOUE, Santa Clara, CA (US)

(72) Inventors: Terah Whiting SMILEY, Davis, CA (US); Venkata Mohan KUDAPA, Foster City, CA (US); Joselito SORIANO, Hayward, CA (US); Nathan LEWIS, San Jose, CA (US); Khoi VU, San Jose, CA (US); Matthew Joseph LANOUE, Santa Clara, CA (US)

(21) Appl. No.: 19/248,330

(22) Filed: Jun. 24, 2025

Related U.S. Application Data
(60) Provisional application No. 63/669,835, filed on Jul. 11, 2024.

(51) Int. Cl. A61F 2/16

(52) U.S. Cl. CPC A61F 2/1629; A61F 2/1643; A61F 2240/001

(57) ABSTRACT

Disclosed are tunable intraocular lenses and methods of adjusting a cylinder power of an intraocular lens. In one aspect, the intraocular lens can comprise an optic portion. The optic portion can comprise a first polymeric bead located at a first location along an exterior of the optic portion and a second polymeric bead located at a second location along the exterior of the optic portion diametrically opposed to the first location.

### CROSS-REFERENCE TO RELATED APPLICATION

This application claims the benefit of priority to U.S. Provisional Patent Application No. 63/669,835 filed on Jul. 11, 2024, the content of which is incorporated herein by reference in its entirety.

### TECHNICAL FIELD

The present disclosure relates generally to the field of ophthalmic lenses, and, more specifically, to tunable intraocular lenses and methods of adjusting a cylinder power of an intraocular lens.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the crystalline lens matrix from the patient's lens capsule and replaces it with an intraocular lens (IOL). Traditional IOLs provide one or more selected focal lengths that allow the patient to have distance vision. However, after cataract surgery, patients with traditional IOLs often require glasses or other corrective eyewear for certain activities since the eye can no longer undertake accommodation (or change its optical power) to maintain a clear image of an object or focus on an object as its distance varies.

Newer IOL such as fluid-filled adjustable IOLs, allow the eye to regain at least some focusing ability. These types of adjustable IOLs use forces available in the eye to change some portion of the optical system in order to refocus the eye on distant or near targets.

However, the manufacturing process for certain newer IOLs can be complicated and require extreme precision. On some occasions, the manufacturing process can yield a lens exhibiting cylinder power that exceeds acceptable thresholds or tolerances.

Therefore, a solution is needed which can adjust the cylinder power of a lens at the later stages of the manufacturing process. Such a solution should also not be overly complicated and should be cost-effective.

### SUMMARY

Disclosed herein are tunable intraocular lenses and methods of adjusting a cylinder power of an intraocular lens. The intraocular lens can comprise an optic portion exhibiting cylinder power. The optic portion can comprise an anterior element including an anterior lateral surface along an exterior periphery of the anterior element and a posterior element. The intraocular lens can also comprise a first polymeric bead located at a first location along the anterior lateral surface and a second polymeric bead located at a second location along the anterior lateral surface. The first location can be diametrically opposed to the second location.

Also disclosed is a method of reducing a cylinder power of an intraocular lens. The method can comprise applying a first polymeric bead to a first location along an anterior lateral surface of an anterior element of an optic portion of the intraocular lens and applying a second polymeric bead to a second location along the anterior lateral surface. The anterior lateral surface can be located along an exterior periphery of the anterior element. The first location can be diametrically opposed to the second location. The method can further comprise curing the intraocular lens with the first polymeric bead applied at the first location and the second polymeric bead applied at the second location.

In some embodiments, the optic portion can have a flat cylinder axis and a steep cylinder axis oriented substantially orthogonal to the flat cylinder axis. In certain embodiments, the first location, where the first polymeric bead is located, and the second location, where the second polymeric bead is located, can both be located along the steep cylinder axis. For example, the first location and the second location can be at opposite ends of the steep cylinder axis.

In other embodiments, the first location and the second location can be located along an axis that is oriented at an oblique angle with respect to the flat cylinder axis. In these embodiments, the first location and the second location are not located along the steep cylinder axis.

In some embodiments, the flat cylinder axis can be measured by wavefront aberrometry. In certain embodiments, the flat cylinder axis can be oriented at an oblique angle with respect to a midline substantially bisecting the optic portion.

In some embodiments, the anterior element can further comprise an anterior outer surface disposed anterior to the anterior lateral surface. At least part of the first polymeric bead can also be located anterior to the first location along a first rim portion of the anterior outer surface and at least part of the second polymeric bead can also be located anterior to the second location along a second rim portion of the anterior outer surface.

In some embodiments, the anterior outer surface can meet the anterior lateral surface at a circular edge. The circular edge can comprise a first edge location and a second edge location diametrically opposed to the first edge location. The first polymeric bead can cover the first edge location and the second polymeric bead can cover the second edge location.

In some embodiments, at least one of the first polymeric bead and the second polymeric bead can have a bead volume of between about 0.0015 microliters (μL) and about 0.05 μL. In certain embodiments, at least one of the first polymeric bead and the second polymeric bead can have a bead size including a bead diameter and a bead height. The bead diameter can be between about 0.2 mm and 0.5 mm. The bead height can be between about 0.05 mm to 0.25 mm.

In some embodiments, at least one of the first polymeric bead and the second polymeric bead can be made of a formulation including one or more cross-linkable polymers and a reactive acrylic monomer diluent.

In some embodiments, the first polymeric bead can be applied and then cured to the anterior element by UV light and the second polymeric bead can be applied and then cured to the anterior element by UV light.

In some embodiments, curing the first polymeric bead to the anterior element and curing the second polymeric bead to the anterior element can reduce the cylinder power of the optic portion by between about 0.1 diopters to 0.5 diopters.

In other embodiments, curing the first polymeric bead to the anterior element and curing the second polymeric bead to the anterior element can adjust the cylinder power and/or a location of the intended cylinder of the optic portion.

Curing the first polymeric bead and the second polymeric bead can shrink the polymeric beads and add tension along an axis at which the polymeric beads are placed.

In some embodiments, the intraocular lens can be an accommodating intraocular lens.

In other embodiments, the intraocular lens can be a fluid-tunable non-accommodating intraocular lens.

Also disclosed is a method of adjusting a cylinder power of an intraocular lens. The method can comprise determining an orientation of a steep cylinder axis of the intraocular lens, applying a first polymeric bead to an exterior of the intraocular lens at a first location at a first end of the steep cylinder axis, and applying a second polymeric bead to the exterior of the intraocular lens at a second location at a second end of the steep cylinder axis opposite the first end.

In some embodiments, the method can further comprise measuring the cylinder power of the intraocular lens, applying a third polymeric bead to the exterior of the intraocular lens, and applying a fourth polymeric bead to the exterior of the intraocular lens.

In some embodiments, the intraocular lens can have a flat cylinder axis substantially orthogonal to the steep cylinder axis. The method can further comprise first determining an orientation of the flat cylinder axis and then determining the orientation of the steep cylinder axis relative to the flat cylinder axis.

In some embodiments, the method can also comprise determining the orientation of the flat cylinder axis by wavefront aberrometry. The flat cylinder axis can be oriented at an oblique angle with respect to a midline substantially bisecting the intraocular lens.

In some embodiments, the intraocular lens can comprise an optic portion comprising an anterior element comprising an anterior lateral surface along an exterior periphery of the anterior element. The first location and the second location can be located along the anterior lateral surface.

In some embodiments, the anterior element can further comprise an anterior outer surface disposed anterior to the anterior lateral surface. The method can further comprise applying a portion of the first polymeric bead to a first rim portion of the anterior outer surface and applying a portion of the second polymeric bead to a second rim portion of the anterior outer surface.

In some embodiments, the anterior outer surface can meet the anterior lateral surface at a circular edge. The circular edge can comprise a first edge location and a second edge location diametrically opposed to the first edge location. The method can further comprise covering the first edge location when applying the first polymeric bead and covering the second edge location when applying the second polymeric bead.

In some embodiments, at least one of the first polymeric bead and the second polymeric bead can have a bead volume of between about 0.0015 μL and about 0.05 μL.

In some embodiments, at least one of the first polymeric bead and the second polymeric bead can have a bead size including a bead diameter and a bead height. The bead diameter can be between about 0.2 mm and 0.5 mm. The bead height can be between about 0.05 mm to 0.25 mm.

In some embodiments, at least one of the first polymeric bead and the second polymeric bead can be made of a formulation including one or more cross-linkable polymers and a reactive acrylic monomer diluent.

In some embodiments, the method can further comprise curing the first polymeric bead and the second polymeric bead to the exterior of the intraocular lens by UV light.

In some embodiments, curing the first polymeric bead and the second polymeric bead can reduce a cylinder power of the intraocular lens by between about 0.1 diopters to 0.5 diopters.

In other embodiments, curing the first polymeric bead and the second polymeric bead can adjust the cylinder power and/or a location of the intended cylinder of the optic portion.

Curing the first polymeric bead and the second polymeric bead can shrink the polymeric beads and add tension along an axis at which the polymeric beads are placed.

In some embodiments, the intraocular lens can be an accommodating intraocular lens.

In other embodiments, the intraocular lens can be a fluid-tunable non-accommodating intraocular lens.

### BRIEF DESCRIPTION OF THE DRAWINGS

FIG. 1A illustrates a top plan view of one embodiment of an intraocular lens.

FIG. 1B illustrates an exploded view of the ophthalmic lens.

FIG. 1C illustrates an exploded view of another embodiment of an intraocular lens.

FIG. 2A illustrates part of the intraocular lens with a first polymeric bead located along an anterior exterior portion of the intraocular lens and a second polymeric bead located along the anterior exterior portion of the intraocular lens.

FIG. 2B is a close-up view of one of the polymeric beads of the intraocular lens.

FIG. 3A is a top plan view of the intraocular lens with one of the haptics removed for ease of viewing.

FIG. 3B is a side view of the intraocular lens with one of the haptics removed for ease of viewing.

FIG. 4A is a top plan view of an anterior element of the intraocular lens with one type of polymeric beads applied to an exterior of the anterior element.

FIG. 4B is a side view of the anterior element of FIG. 4A.

FIG. 5A is a top plan view of an anterior element of the intraocular lens with another type of polymeric beads applied to an exterior of the anterior element.

FIG. 5B is a side view of the anterior element of FIG. 5A.

FIG. 6A is a wavefront map of one embodiment of the intraocular lens with polymeric beads applied to an exterior of the intraocular lens.

FIG. 6B is a wavefront map of another embodiment of the intraocular lens with multiple polymeric beads applied to an exterior of the intraocular lens.

FIG. 7 is a flowchart illustrating one embodiment of a method of reducing a cylinder power of an intraocular lens.

FIG. 8 is a flowchart illustrating one embodiment of a method of adjusting a cylinder power of an intraocular lens.

### DETAILED DESCRIPTION

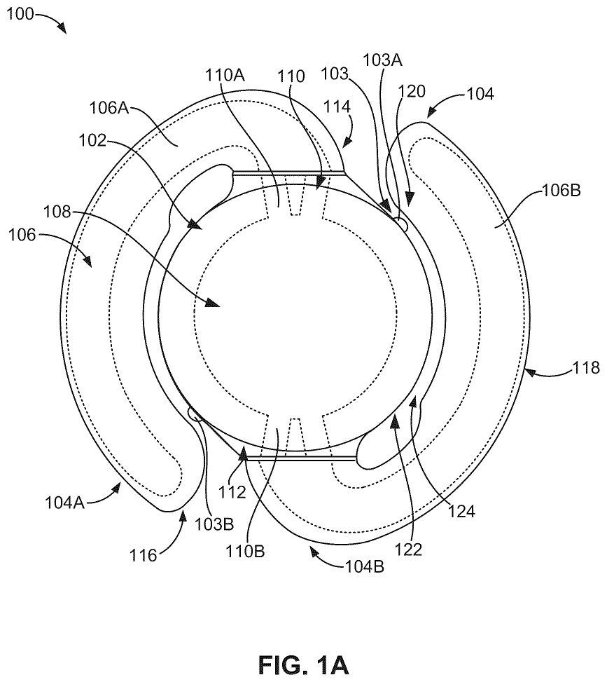

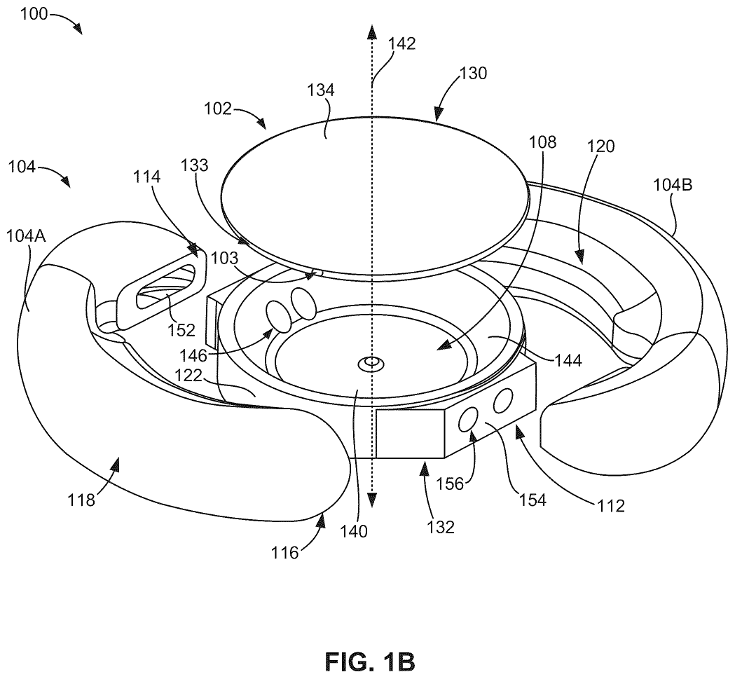

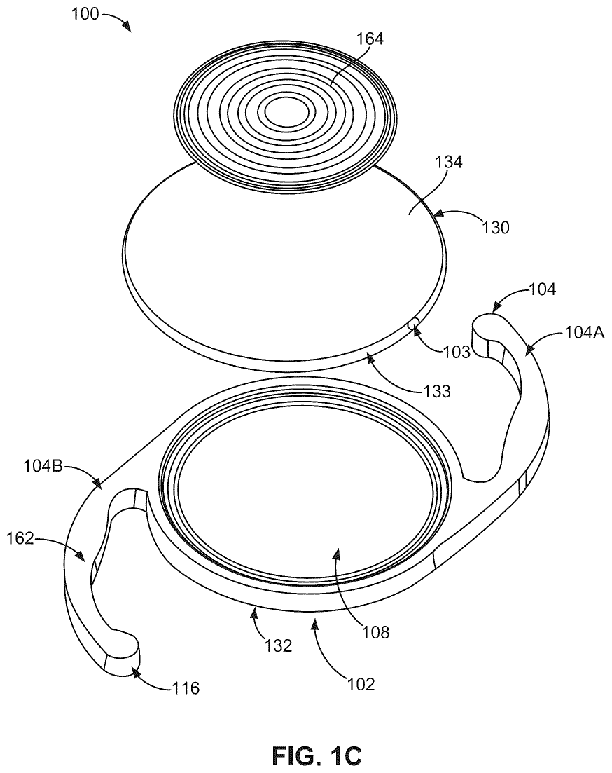

FIG. 1A illustrates a top plan view of one embodiment of an intraocular lens 100. The intraocular lens 100 can be implanted within a subject to correct for defocus aberration, corneal astigmatism, spherical aberration, or a combination thereof.

The intraocular lens 100 can comprise an optic portion 102 and one or more haptics 104 including a first haptic 104A and a second haptic 104B coupled to and extending peripherally from the optic portion 102. For example, the intraocular lens 100 can be positioned within a native capsular bag in which a native lens has been removed. When the intraocular lens 100 is implanted within the native capsular bag, the optic portion 102 can be adapted to refract light that enters the eye onto the retina.

In some embodiments, the haptics 104 can be coupled to and adhered to the optic portion 102. For example, the haptics 104 can be adhered to the optic portion 102 after each is formed separately. In other embodiments, the intraocular lens 100 can be a one-piece lens such that the haptics 104 are connected to and extend from the optic portion 102. In this example embodiment, the haptics 104 are formed along with the optic portion 102 and are not adhered or otherwise coupled to the optic portion 102 in a subsequent step.

The intraocular lens 100 can have an uncompressed haptic length as measured from a haptic distal end of the first haptic 104A to the haptic distal end of the second haptic 104B. The uncompressed haptic length can be between about 8.0 mm and about 14.0 mm. For example, the uncompressed haptic length can be about 10.0 mm.

In some embodiments, the optic portion 102 of the intraocular lens 100 can have an optic portion diameter. The optic portion diameter can be between about 4.0 mm and 8.0 mm. For example, the optic portion diameter can be about 6.0 mm.

The intraocular lens 100 can also comprise a plurality of polymeric beads 103 or polymeric dots or droplets located on an exterior of the optic portion 102 intraocular lens 100. For example, the polymeric beads 103 or polymeric dots can comprise a first polymeric bead 103A and a second polymeric bead 103B.

The polymeric beads 103 can be cross-linkable polymeric beads that can shrink in response to light energy (e.g., UV light) directed at the polymeric beads 103.

In certain embodiments, the polymeric beads 103 can also be referred to as adhesive beads or adhesive dots.

The first polymeric bead 103A can be located at one position along an outer peripheral surface 122 of the optic portion 102 and the second polymeric bead 103B can be located at another position along the outer peripheral surface 122. In some embodiments, the first polymeric bead 103A can be located diametrically opposed to the second polymeric bead 103B. The polymeric beads 103 will be discussed in more detail in later sections.

In some embodiments, the intraocular lens 100 can be a fluid-filled IOL such as an accommodating IOL (or “AIOL”). As will be discussed in more detail in later sections, the intraocular lens 100 can also be a fluid-tunable non-accommodating intraocular lens (see, e.g., FIG. 1C).

When the intraocular lens 100 is an AIOL, one or more haptics 104 can be configured to engage the capsular bag and be adapted to deform in response to ciliary muscle movement (e.g., muscle relaxation, muscle contraction, or a combination thereof) in connection with capsular bag reshaping. Each of the haptics 104 can comprise a haptic fluid lumen 106 (shown in broken or phantom lines) extending through at least part of the haptic 104. For example, the first haptic 104A can comprise a first haptic fluid lumen 106A extending through at least part of the first haptic 104A and the second haptic 104B can comprise a second haptic fluid lumen 106B extending through at least part of the second haptic 104B. The haptic fluid lumen 106 (e.g., any of the first haptic fluid lumen 106A or the second haptic fluid lumen 106B) can be in fluid communication with or fluidly connected to an optic fluid chamber 108 within the optic portion 102.

The optic fluid chamber 108 and the haptic fluid lumen(s) 106 can comprise a fluid. A base power of the optic portion 102 can be configured to change based on an internal fluid pressure within the fluid-filled optic fluid chamber 108. The base power of the optic portion 102 can be configured to increase or decrease as fluid enters or exits the fluid-filled optic fluid chamber 108. For example, the base power of the optic portion 102 can be configured to decrease as fluid exits or is drawn out of the fluid-filled optic fluid chamber 108 into the haptic fluid lumen(s) 106. Also, for example, the base power of the optic portion 102 can be configured to increase as fluid enters the fluid-filled optic fluid chamber 108 from the haptic fluid lumen(s) 106.

The optic fluid chamber 108 can be in fluid communication with the one or more haptic fluid lumens 106 through one or more fluid channels 110. The fluid channels 110 can be conduits or passageways fluidly connecting the optic fluid chamber 108 to the haptic fluid lumens 106. The fluid channels 110 can be spaced apart from one another. For example, a pair of fluid channels 110 can be spaced apart between about 0.1 mm to about 1.0 mm. In some embodiments, each of the fluid channels 110 can have a diameter of between about 0.4 mm to about 0.6 mm.

The haptics 104 can be coupled to the optic portion 102 at a reinforced portion. The reinforced portion can serve as a haptic-optic interface 112. The pair of fluid channels 110 can be defined or formed within part of the reinforced portion.

As shown in FIG. 1A, the optic fluid chamber 108 can be in fluid communication with the first haptic fluid lumen 106A through a first pair of fluid channels 110A. The optic fluid chamber 108 can also be in fluid communication with the second haptic fluid lumen 106B through a second pair of fluid channels 110B.

In some embodiments, the first pair of fluid channels 110A and the second pair of fluid channels 110B can be positioned substantially on opposite sides of the optic portion 102. The first pair of fluid channels 110A can be positioned substantially diametrically opposed to the second pair of fluid channels 110B. The first pair of fluid channels 110A and the second pair 11 of fluid channels 110B can extend or be defined through part of the optic portion 102. The first pair of fluid channels 110A and the second pair of fluid channels 110B can extend or be defined through a posterior element 132 of the optic portion 102 (see, e.g., FIG. 1B).

FIG. 1A also illustrates that each of the haptics 104 (e.g., any of the first haptic 104A or the second haptic 104B) can have a proximal attachment end 114 and a distal free end 116. A haptic fluid port 152 (see, e.g., FIG. 1B) can be defined at the proximal attachment end 114 of the haptic 104. The haptic fluid port 152 can serve as an opening of the haptic fluid lumen 106. Fluid within the haptic fluid lumen 106 can flow out of the haptic fluid lumen 106 through the haptic fluid port 152 and into the optic fluid chamber 108 via the fluid channels 110 when the haptic 104 is coupled to the optic portion 102. Similarly, fluid within the optic fluid chamber 108 can flow out of the optic fluid chamber 108 through the pair of fluid channels 110 and into the haptic fluid lumen 106 through the haptic fluid port 152.

Each of the haptics 104 can comprise a radially-outer haptic lumen wall 118 and a radially-inner haptic lumen wall 120. The radially-outer haptic lumen wall 118 (also referred to as a radially-outer lateral wall of the haptic 104) can be configured to face and contact an inner surface of a patient's capsular bag when the intraocular lens 100 is implanted within the capsular bag. The radially-inner haptic lumen wall 120 (also referred to as a radially-inner lateral wall of the haptic 104) can be configured to face an outer peripheral surface 122 of the optic portion 102.

As previously discussed, the intraocular lens 100 can be implanted or introduced into a patient's capsular bag after a native lens has been removed from the capsular bag. The patient's capsular bag is connected to zonule fibers which are connected to the patient's ciliary muscles. The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flatter (in the anterior-to-posterior direction), which reduces the power of the lens, allowing for distance vision. In this configuration, the patient's native lens is said to be in a disaccommodated state or undergoing disaccommodation.

When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved (e.g., the anterior part of the lens becomes more curved), which gives the lens more power, allowing the eye to focus on near objects. In this configuration, the patient's native lens is said to be in an accommodated state or undergoing accommodation.

In embodiments where the intraocular lens 100 is an AIOL, the radially-outer haptic lumen wall 118 of the implanted AIOL can directly engage with or be in physical contact with the portion of the capsular bag that is connected to the zonules or zonule fibers. Therefore, the radially-outer haptic lumen wall 118 of the AIOL can be configured to respond to capsular bag reshaping forces that are applied radially when the zonules relax and stretch as a result of ciliary muscle movements.

For example, when the ciliary muscles contract, the peripheral region of the elastic capsular bag reshapes and applies radially inward forces on the radially-outer haptic lumen wall 118 of each of the haptics 104. When the intraocular lens 100 is an AIOL, the radially-outer haptic lumen wall 118 can deform or otherwise change shape and this deformation or shape-change can cause the volume of the haptic fluid lumen 106 to decrease. When the volume of the haptic fluid lumen 106 decreases, the fluid within the haptic fluid lumen 106 is moved or pushed into the optic fluid chamber 108. The optic portion 102 of the AIOL can change shape in response to fluid entering the optic fluid chamber 108 from the haptic fluid lumen 106. This can increase the base power or base spherical power of the AIOL and allow a patient with the AIOL implanted within the eye of the patient to focus on near objects. In this state, the adjustable AIOL can be considered to have undergone accommodation.

When the ciliary muscles relax, the peripheral region of the elastic capsular bag is stretched radially outward and the capsular bag elongates and more room is created within the capsular bag. The radially-outer haptic lumen wall 118 of the haptics 104 can be configured to respond to this capsular bag reshaping by returning to its non-deformed or non-stressed 9 configuration. This causes the volume of the haptic fluid lumen 106 to increase or return to its non-deformed volume. This increase in the volume of the haptic fluid lumen 106 can cause the fluid within the optic fluid chamber 108 to be drawn out or otherwise flow out of the optic fluid chamber 108 and back into the haptic fluid lumen 106. Fluid moves out of the optic fluid chamber 108 into the haptic fluid lumen 106 through the same fluid channels 110 formed within the optic portion 102.

The optic portion 102 of the AIOL can change shape in response to fluid exiting the optic fluid chamber 108 and into the haptic fluid lumen 106. This can decrease the base power or base spherical power of the AIOL and allow a patient with the AIOL implanted within the eye of the patient to focus on distant objects or provide for distance vision. In this state, the AIOL can be considered to have undergone disaccommodation.

When the intraocular lens 100 is an AIOL, the radially-outer haptic lumen walls 118 of the haptics 104 can be made thinner than the radially-inner haptic lumen walls 120 to allow the haptics 104 to maintain a high degree of sensitivity to radial forces applied to an equatorial region of the haptics 104 by capsular bag reshaping as a result of ciliary muscle movements. The radially-inner haptic lumen walls 120 of the haptics 104 can be designed to be thicker or bulkier than the radially-outer haptic lumen walls 118 to provide the haptics 104 with stiffness or resiliency in the anterior-to-posterior direction. In certain embodiments, the radially-inner 27 haptic lumen wall 120 can taper in shape as the radially-inner haptic lumen wall 120 gets closer to the optic portion 102. When designed in this manner, the haptics 104 can be less sensitive to capsular bag forces applied in the anterior-to-posterior direction. For example, when capsular bag forces are applied to the haptics 104 in the anterior-to-posterior direction, less fluid movement occurs between the haptic fluid lumens 106 and the optic fluid chamber 108 than when forces are applied in the radial direction. Since less fluid movement occurs, less changes in the base power of the AIOL occur.

Examples of AIOLs are discussed in the following U.S. patent publications: U.S. Pat. Pub. No. 2018/0153682 and in the following issued U.S. Pat. Nos. 11,744,697; 11,660,182; 11,622,850; 11,426,270; 10,433,949; 10,299,913; 10,195,020; and 8,968,396, the contents of which are incorporated herein by reference in their entireties.

As will be discussed in more detail in relation to FIG. 1C, the intraocular lens 100 can also be a fluid-tunable non-accommodating IOL or a non-accommodating static-focus adjustable IOL. Examples of fluid-tunable non-accommodating IOLs or non-accommodating static-focus adjustable IOLs are discussed in U.S. Pat. No. 11,471,272, the content of which is incorporated herein by reference in its entirety.

In some embodiments, the intraocular lens 100 can be designed such that a gap 124 or void space radially separates the radially-inner haptic lumen wall 120 of the haptic 104 from the outer peripheral surface 122 of the optic portion 102.

In some embodiments, the fluid within the optic fluid chamber 108 and the haptic fluid lumen(s) 106 can be an oil. More specifically, in certain embodiments, the fluid within the optic fluid chamber 108 and the haptic fluid lumen(s) 106 can be a silicone oil or fluid. For example, the fluid can be a silicone oil made in part of a diphenyl siloxane. In other embodiments, the fluid can be a silicone oil made in part of a ratio of two dimethyl siloxane units to one diphenyl siloxane unit. More specifically, in some embodiments, the fluid can be a silicone oil made in part of diphenyltetramethyl cyclotrisiloxane or a copolymer of diphenyl siloxane and dimethyl siloxane. In further embodiments, the fluid can be a silicone oil comprising branched polymers.

The fluid (e.g., the silicone oil) can be index-matched with a lens body material used to make the optic portion 102. When the fluid is index-matched with the lens body material, the entire optic portion 102 containing the fluid can act as a single lens. For example, the fluid can be selected so that it has a refractive index of between about 1.48 and 1.53 (or between about 1.50 and 1.53). In some embodiments, the fluid (e.g., the silicone oil) can have a polydispersity index of between about 1.2 and 1.3. In other embodiments, the fluid (e.g., the silicone oil) can have a polydispersity index of between about 1.3 and 1.5. In other embodiments, the fluid (e.g., the silicone oil) can have a polydispersity index of between about 1.1 and 1.2. Other example fluids are described in U.S. Patent Publication No. 2018/0153682, which is herein incorporated by reference in its entirety.

FIG. 1B illustrates an exploded view of the intraocular lens 100. The optic portion 102 of the intraocular lens 100 can comprise an anterior element 130 and a posterior element 132. A fluid-filled optic fluid chamber 108 can be defined in between the anterior element 130 and the posterior element 132.

The anterior element 130 can comprise an anterior outer surface 134 and an anterior inner surface opposite the anterior outer surface 134. The posterior element 132 can comprise a posterior outer surface and a posterior inner surface 140 opposite the posterior outer surface. Any of the anterior outer surface 134, the posterior optical surface, or a combination thereof can be considered and referred to as an external optical surface. The anterior inner surface and the posterior inner surface 140 can face the optic fluid chamber 108. At least part of the anterior inner surface and at least part of the posterior inner surface 140 can serve as chamber walls of the optic fluid chamber 108.

As shown in FIGS. 1B, the optic portion 102 can have an optical axis 142 extending in an anterior-to-posterior direction through a center of the optic portion 102. The optical axis 142 can extend through the centers of both the anterior element 130 and the posterior element 132.

The thickness of the anterior element 130 can be greater at or near the optical axis 142 than at the periphery of the anterior element 130. In some embodiments, the thickness of the anterior element 130 can increase gradually from the periphery of the anterior element 130 toward the optical axis 142.

In certain embodiments, the thickness of the anterior element 130 at or near the optical axis 142 can be between about 0.45 mm and about 0.55 mm. In these and other embodiments, the thickness of the anterior element 130 near the periphery can be between about 0.20 mm and about 0.40 mm. Moreover, the anterior inner surface of the anterior element 130 can have less curvature or be flatter than the anterior outer surface 134.

The thickness of the posterior element 132 can be greater at or near the optical axis 142 than portions of the posterior element 132 radially outward from the optical axis 142 but prior to reaching a raised periphery 144 of the posterior element 132. The thickness of the posterior element 132 can gradually decrease from the optical axis 142 to portions radially outward from the optical axis 142 (but prior to reaching the raised periphery 144). As shown in FIG. 1B, the thickness of the posterior element 132 can increase once again from a radially inner portion of the raised periphery 144 to a radially outer portion of the raised periphery 144.

In certain embodiments, the thickness of the posterior element 132 at or near the optical axis 142 can be between about 0.45 mm and about 0.55 mm. In these and other embodiments, the thickness of the posterior element 132 radially outward from the optical axis 142 (but prior to reaching the raised periphery 144) can be between about 0.20 mm and about 0.40 mm. The thickness of the posterior element 132 near the radially outer portion of the raised periphery 144 can be between about 1.00 mm and 1.15 mm. Moreover, the posterior inner surface 140 of the posterior element 132 can have less curvature or be flatter than the posterior optical surface.

FIG. 1B also illustrates that each of the haptics 104 (e.g., any of the first haptic 104A or the second haptic 104B) can have a proximal attachment end 114 and a closed distal free end 116. A haptic fluid port 152 can be defined at the proximal attachment end 114 of the haptic 104. The haptic fluid port 152 can serve as a chamber opening of the haptic fluid lumen 106. Fluid within the haptic fluid lumen 106 can flow out of the haptic fluid lumen 106 through the haptic fluid port 152 and into the optic fluid chamber 108 via the pair of fluid channels 110 when the haptic 104 is coupled to the optic portion 102. Similarly, fluid within the optic fluid chamber 108 can flow out of the optic fluid chamber 108 through the pair of fluid channels 110 and into the haptic fluid lumen 106 through the haptic fluid port 152. A pair of outer apertures 156 and inner apertures 146 can serve as ends of the fluid channels 110.

As shown in FIG. 1B, each of the haptics 104 can be coupled to the optic portion 102 at the haptic-optic interface 112. More specifically, the proximal attachment end 114 can be coupled to the protruding outer surface 154 of the posterior element 132. The protruding outer surface 154 can also be referred to as a “landing” or “haptic attachment landing.” The protruding outer surface 154 can extend out radially from an outer peripheral surface 122 of the optic portion 102. For example, the protruding outer surface 154 can extend out radially from an outer peripheral surface 122 of the posterior element 132 of the optic portion 102. The protruding outer surface 154 can extend out radially from the outer peripheral surface 122 between about 10 microns and 1.0 mm or between about 10 microns and 500 microns.

The proximal attachment end 114 can have a substantially flat surface to adhere or otherwise couple to a substantially flat surface of the protruding outer surface 154. When the proximal attachment end 114 is coupled to the protruding outer surface 154, the haptic fluid port 152 can surround the outer apertures 156 of the fluid channels 110. The haptics 104 can be coupled or adhered to the optic portion 102 via biocompatible adhesives. In some embodiments, the adhesives can be the same adhesives used to couple or adhere the anterior element 130 to the posterior element 132.

FIG. 1B also illustrates that the anterior element 130 can have an anterior lateral surface 133 disposed along an exterior periphery of the anterior element 130. As shown in FIG. 1B, at least one of the polymeric beads 103 can be located along the anterior lateral surface 133.

FIG. 1C illustrates an exploded perspective view of another embodiment of an intraocular lens 100. The intraocular lens 100 shown in FIG. 1C can be a fluid-tunable non-accommodating intraocular lens.

The intraocular lens 100 can comprise an optic portion 102 and one or more haptics 104 extending from the optic portion 102. The haptics 104 can comprise a first haptic 104A and a second haptic 104B extending peripherally from or coupled to the optic portion 102. Each of the haptics 104 can comprise a kink 162 or bend defined along an arm of the haptic 104. The kink 162 or bend can allow the haptic 104 to compress or flex. Each of the haptics 104 can terminate at a free or unconnected haptic distal end 116.

For example, the intraocular lens 100 can be a one-piece lens such that the haptics 104 are connected to and extend from the optic portion 102. In other embodiments, the haptics 104 are coupled to and adhered to the optic portion 102. For example, the haptics 104 can be adhered to the optic portion 102 after each is formed separately.

The optic portion 102 can comprise an anterior element 130, a posterior element 132, and an optic chamber 108 defined in between the anterior element 130 and the posterior element 132. The optic chamber 108 can be filled with a fluid.

The anterior element 130 shown in FIG. 1C can have an anterior lateral surface 133 disposed along an exterior periphery of the anterior element 130. One or more polymeric beads 103 can be located along the anterior lateral surface 133 of the anterior element 130.

In some embodiments, the fluid within the optic chamber 110 can be an oil. More specifically, in certain embodiments, the fluid within the optic chamber 110 can be a silicone oil.

The anterior element 130 can comprise an anterior outer surface 134. The anterior outer surface 134 can comprise a unique lens surface profile 164 or pattern defined on the anterior outer surface 134.

In some embodiments, the lens surface profile 164 can comprise a central diffractive area or structure comprising a plurality of diffractive zones or steps. In these and other embodiments, the widths of the diffractive zones can decrease in a radially outward manner such that zone widths at a periphery of the lens are smaller than zone widths near a central portion of the lens.

In certain embodiments, the lens surface profile 164 can split light into multiple foci or focal points. In these embodiments, the intraocular lens 100 can be considered a multifocal IOL or an adjustable multifocal IOL.

In some embodiments, the lens surface profile 164 can be configured to split light into two focal points (e.g., allowing for near and distant vision). In these embodiments, the intraocular lens 100 can be considered a bifocal IOL or an adjustable bifocal IOL.

The lens surface profile 164 can also be configured to split light into three focal points (e.g., allowing for near, intermediate, and distant vision). In these embodiments, the intraocular lens 100 can be considered a trifocal IOL or an adjustable trifocal IOL.

In other embodiments not shown in FIG. 1C, the external optical surface can have a uniformly curved (e.g., a spherical) lens surface or an aspherical lens surface providing focusing power for a single distance. In these embodiments, the intraocular lens 100 can be considered a monofocal IOL or an adjustable monofocal IOL.

In additional embodiments not shown in FIG. 1C, the anterior outer surface 134 can have a lens surface profile or pattern configured to provide an extended depth of focus or a single elongated focal point. In these embodiments, the intraocular lens 100 can be considered an extended depth of focus (EDOF) IOL or an adjustable EDOF IOL.

Moreover, any of the monofocal IOLs, the multifocal IOLs, or the EDOF IOLs can comprise a toric lens profile.

The intraocular lens 100 can have an uncompressed haptic length as measured from a haptic distal end of the first haptic 104A to the haptic distal end of the second haptic 104B. The uncompressed haptic length can be between about 12.0 mm and about 14.0 mm. For example, the uncompressed haptic length can be about 13.0 mm.

In some embodiments, the optic portion 102 of the intraocular lens 100 can have an optic portion diameter. The optic portion diameter can be between about 5.0 mm and 8.0 mm. For example, the optic portion diameter can be about 6.0 mm.

In certain embodiments, the intraocular lens 100 (either the intraocular lens 100 shown in FIGS. 1A and 1B or the intraocular lens 100 shown in FIG. 1C) can be made of a lens material. In some embodiments, the lens material can be a soft polymeric material. For example, the lens material can be a hydrophobic acrylic material.

For example, the lens material can be made in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably unless otherwise specified.

The cross-linked copolymer used to make the lens material can comprise an alkyl acrylate or methacrylate in the amount of about 3% to 20% (wt %), a fluoro-alkyl acrylate or fluoro-alkyl methacrylate in the amount of about 10% to 35% (wt %), and a phenyl-alkyl acrylate in the amount of about 50% to 80% (wt %). In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate. More specifically, the cross-linked copolymer used to make the lens material can comprise n-butyl acrylate in the amount of about 3% to 20% (wt %) (e.g., between about 12% to 16%), trifluoroethyl methacrylate in the amount of about 10% to 35% (wt %) (e.g., between about 17% to 25%), and phenylethyl acrylate in the amount of about 50% to 80% (wt %) (e.g., between about 64% to 67%).

The final composition of the cross-linked copolymer used to make the lens body material can also comprise a cross-linker or cross-linking agent, such as ethylene glycol dimethacrylate (EGDMA), and a hydroxyl-functional acrylic monomer (hydroxyethyl acrylate (HEA) or hydroxyethyl methacrylate (HEMA)). For example, the final composition of the cross-linked copolymer used to make the lens material can also comprise a cross-linker or cross-linking agent (e.g., EGDMA) in the amount of about 1.0%. The final composition of the cross-linked copolymer used to make the lens body material can also comprise an initiator or initiating agent (e.g., Perkadox 16, Darocur, etc.) and a UV absorber.

The haptic(s) 104 can comprise or be made in part of a haptic material. In some embodiments, the haptic material can be a soft polymeric material. For example, the haptic material can be a hydrophobic acrylic material.

For example, the haptic material can comprise or be made in part of a cross-linked copolymer comprising a copolymer blend. The copolymer blend can comprise an alkyl acrylate, a fluoro-alkyl acrylate, and a phenyl-alkyl acrylate. For example, the cross-linked copolymer used to make the haptic material can comprise an alkyl acrylate in the amount of about 10% to 25% (wt %), a fluoro-alkyl acrylate in the amount of about 10% to 35% (wt %), and a phenyl-alkyl acrylate in the amount of about 50% to 80% (wt %). In some embodiments, the cross-linked copolymer used to make the haptic material can comprise n-butyl acrylate in the amount of about 10% to 25% (wt %) (e.g., between about 19% to about 23%), trifluoroethyl methacrylate in the amount of about 10% to 35% (wt %) (e.g., between about 14% to about 18%), and phenylethyl acrylate in the amount of about 50% to 80% (wt %) (e.g., between about 58% to about 62%). The final composition of the cross-linked copolymer used to make the haptic material can also comprise a cross-linker or cross-linking agent, such as EGDMA, in the amount of about 1.0%. The final composition of the cross-linked copolymer used to make the haptic material can also comprise a number of photoinitiators or photoinitiating agents (e.g., camphorquinone, 1-phenyl-1,2-propanedione, 2-ethylhexyl-4-(dimenthylamino)benzoate, etc.).

In some embodiments, the refractive index of the lens material can be between about 1.48 and about 1.53. In certain embodiments, the refractive index of the lens material can be between about 1.50 and about 1.53 (e.g., about 1.5178).

In these and other embodiments, at least part of the haptic(s) 104 and/or the optic portion 102 (e.g., the anterior element 130 and/or the posterior element 132) can be made of a composite material. The composite material can comprise an energy-absorbing constituent and a plurality of expandable components. A base power of the optic portion 102 (either the optic portion 102 shown in FIGS. 1A and 1B or the optic portion shown in FIG. 1C) can be configured to change in response to an external energy (e.g., laser light) directed at the composite material. Depending on where the composite material is placed or positioned within the haptic(s) 104 or the optic portion 102, expansion of the composite material can either increase or decrease the volume of the optic fluid chamber 108 and change the fluid pressure within the optic fluid chamber 108. The optic portion 102 (e.g., the anterior element 130, the posterior element 132, or a combination thereof) can be configured to deform, flex, or otherwise change shape in response to a change in the fluid pressure within the optic fluid chamber 108. The base power of the optic portion can be configured to change between about 0.05 D to about 3.0 D in total in response to pulses of the external energy (e.g., laser light) directed at the composite material.

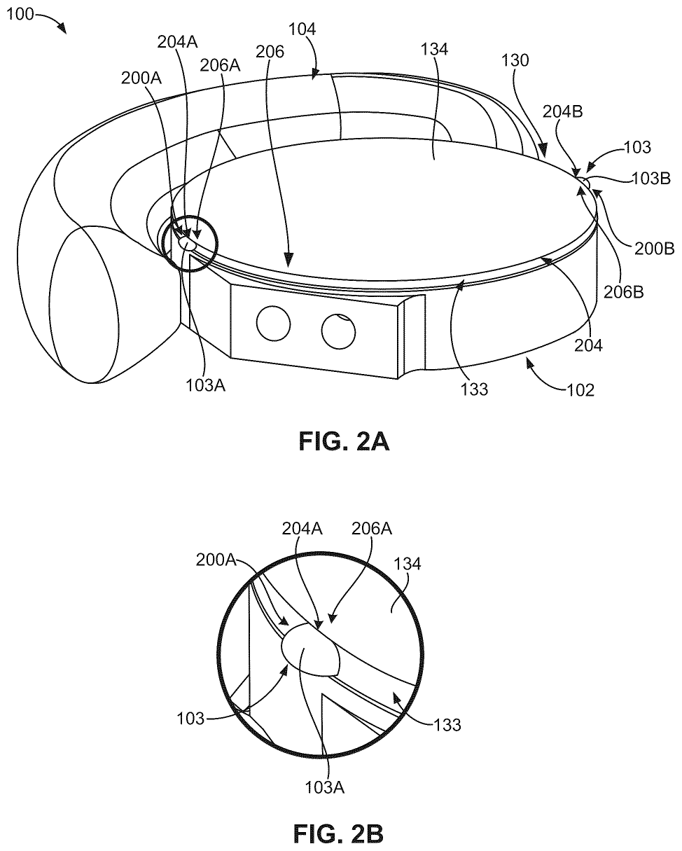

FIG. 2A illustrates one embodiment of the intraocular lens 100 with a first polymeric bead 103A located along an anterior lateral surface 133 of the anterior element 130 and a second polymeric bead 103B also located along the anterior lateral surface 133. One of the haptics 104 of the intraocular lens 100 is not shown in FIG. 2A for ease of viewing.

As shown in FIG. 2A, the intraocular lens 100 can comprise the first polymeric bead 103A located at a first location 200A along the anterior lateral surface 133 and the second polymeric bead 103B located at a second location 200B along the anterior lateral surface 133. The first location 200A can be diametrically opposed to the second location 200B.

The polymeric beads 103 can be applied to the anterior lateral surface 133 of the anterior element 130 of the optic portion 102 to induce additional tensile stress on the intraocular lens 100 to reduce cylinder power along the steep axis of the optic portion 102. For example, the polymeric beads 103 can be applied to the anterior lateral surface 133 to induce additional tensile stress on the optic portion 102 to flatten the radius of curvature along the steep axis.

As previously discussed, on some occasions, the manufacturing process can yield an intraocular lens 100 where the optic portion 102 exhibits cylinder power that is out of specification or exhibits unwanted cylinder power. Applying polymeric beads 103 to the anterior element 130 of the optic portion 102 can ameliorate the effects of this unwanted or out-of-specification cylinder power by reducing or otherwise adjusting the cylinder power.

FIG. 2A also illustrates that the anterior element 130 can further comprise an anterior outer surface 134 disposed anterior to the anterior lateral surface 133. The anterior outer surface 134 can meet the anterior lateral surface 133 at a circular edge 204 extending circumferentially around the anterior element 130.

The anterior outer surface 134 can also comprise a rim portion 206 disposed around a periphery of the anterior outer surface 134 but radially inward of the circular edge 204. The anterior outer surface 134 can further comprise a first rim portion 206A located immediately anterior to the first location 200A and a second rim portion 206B located immediately anterior to the second location 200B.

As will be discussed in more detail in later sections, in some embodiments, at least part of the first polymeric bead 103A or another polymeric bead can be located anterior to the first location 200A along the first rim portion 206A of the anterior outer surface 134 and at least part of the second polymeric bead 103B or another polymeric bead can be located anterior to the second location 200B along the second rim portion 206B of the anterior outer surface 134.

As depicted in FIG. 2A, the circular edge 204 can comprise a first edge location 204A and a second edge location 204B diametrically opposed to the first edge location 204A. The 19 first edge location 204A can be located radially outward of the first rim portion 206A and the second edge location 204B can be located radially outward of the second rim portion 206B.

In some embodiments, the first polymeric bead 103A can be applied to the first edge location 204A and/or the first rim portion 206A and the second polymeric bead 103B can be applied to the second edge location 204B and/or the second rim portion 206B.

In further embodiments, the first polymeric bead 103A can be applied to the first location 200A along the anterior lateral surface 133 and the first rim portion 206A of the anterior outer surface 134 (see, e.g., FIG. 5A and FIG. 5B). In these embodiments, the second polymeric bead 103B can be applied to the second location 200B along the anterior lateral surface 133 and the second rim portion 206B of the anterior outer surface 134 (see, e.g., FIG. 5A and FIG. 5B). Also, in these embodiments, the first polymeric bead 103A can cover the first edge location 204A and the second polymeric bead 103B can cover the second edge location 204B (see, e.g., FIG. 5A and FIG. 5B).

FIG. 2B is a close-up view of the first polymeric bead 103A from FIG. 2A. Although FIG. 2B depicts the first polymeric bead 130A, it should be understood by one of ordinary skill in the art that the polymeric bead shown in FIG. 2A can be any of the polymeric beads 103 applied to the optic portion 102 of the intraocular lens 100.

As illustrated in FIG. 2B, the polymeric bead 103 (e.g., the first polymeric bead 103A or the second polymeric bead 103B) can have a bead shape, a bead size, and a bead volume.

In some embodiments, the bead shape of the polymeric bead 103 can be substantially hemispheroid. In other embodiments, the polymeric bead 103 can be substantially shaped as a truncated or partial spheroid.

In further embodiments, the bead shape of the polymeric bead 103 can be shaped as a hemi-prolate spheroid or a truncated/partial prolate spheroid. In additional embodiments, the bead shape of the polymeric bead 103 can be shaped as a hemi-oblate spheroid or a truncated/partial oblate spheroid.

In certain embodiments, the polymeric bead 103 (e.g., the first polymeric bead 103A or the second polymeric bead 103B) can have a bead size as measured by a bead diameter, at a base of the bead, and a bead height. For example, the bead diameter can be between about 0.2 mm and 0.5 mm. Also, for example, the bead height can be between about 0.05 mm to 0.25 mm.

In some embodiments, the bead diameter can also be between about 0.05 mm and 0.2 mm or between about 0.50 mm and 1.0 mm. The bead height can also be between about 0.01 mm and 0.05 mm or between about 0.25 mm and 0.50 mm.

The polymeric bead 103 (e.g., the first polymeric bead 103A or the second polymeric bead 103B) can have a bead volume. In some embodiments, the bead volume of the polymeric bead 103 can be between about 0.0015 μL and about 0.05 μL. In other embodiments, the bead volume of the polymeric bead 103 can be between about 0.05 μL and 0.10 μL.

In some embodiments, the polymeric beads 103 can be made of a formulation comprising one or more cross-linkable polymers and a reactive acrylic monomer diluent. For example, the monomers of the cross-linkable polymers can comprise trifluoroethyl methacrylate, at least one of butyl acrylate and n-butyl methacrylate, at least one of phenylethyl acrylate and phenylethyl methacrylate, and the reactive acrylic monomer diluent.

In certain embodiments, the reactive acrylic monomer diluent can be between about 23% and about 43% of the formulation. In these embodiments, the monomers of the cross-linkable polymer can be between about 55% and about 75% of the formulation.

As a more specific example, the butyl acrylate or n-butyl methacrylate can be between about 35% and about 45% of the cross-linkable polymer, the phenylethyl acrylate or the phenylethyl methacrylate can be between about 20% and about 40% of the cross-linkable polymer, and the trifluoroethyl methacrylate can be between about 15% and about 30% of the cross-linkable polymer.

The formulation can further comprise a photoinitiator (e.g., about 2% of the formulation). The formulation can also comprise hydroxyl ethyl acrylate.

In some embodiments, the polymeric beads 103 can be applied and then cured to the anterior element 130 by ultraviolet (UV) light.

In some embodiments, the polymeric beads 103, once applied to the anterior element 130, can be cured for between about 30 seconds and 300 seconds. In certain embodiments, curing the polymeric beads 103 (e.g., the first polymeric bead 103A and the second polymeric bead 103B) to the anterior element 130 can reduce the cylinder power of the optic portion 102 by between about 0.1 diopters to 0.5 diopters.

In other embodiments, curing the polymeric beads 103 (e.g., the first polymeric bead 103A and the second polymeric bead 103B) to the anterior element 130 can adjust a cylinder power of the optic portion 102 and/or an angular location of a cylinder axis. For example, an intraocular lens 100 might be designed to have 1 diopter of cylinder located at 90 degrees but this intraocular lens 100 is measured to have 0.8 diopters of cylinder located at 75 degrees. Polymeric beads 103 can then be applied to the anterior element 130 of this intraocular lens 100 to adjust the cylinder power and the angular location of the cylinder to the designed specifications.

In some embodiments, the polymeric beads 103 can shrink between about 1% and 10% (e.g., about 3%) by volume after the curing step. As will be discussed in more detail in the following sections, when the polymeric beads 103 shrink, the polymeric beads 103 can add tension or induce certain tensile stresses on the optic portion 102 along an axis at which the polymeric beads 103 are placed.

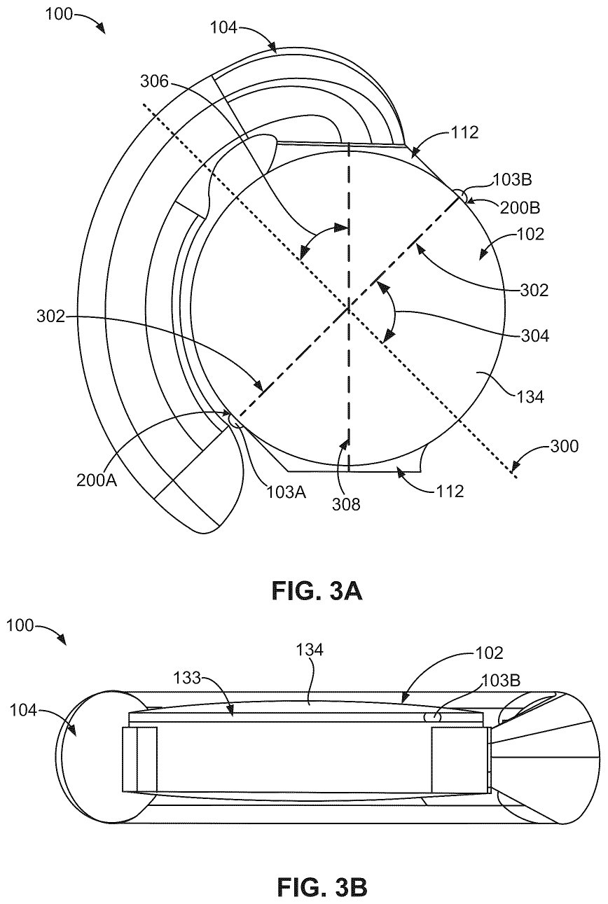

FIG. 3A is a top plan view of the intraocular lens 100 with one of the haptics 104 removed for ease of viewing. FIG. 3B is a side view of the intraocular lens 100 shown in FIG. 3A.

As illustrated in FIG. 3A, the optic portion 102 of the intraocular lens 100 can have a flat cylinder axis 300 and a steep cylinder axis 302 oriented substantially orthogonal or perpendicular to the flat cylinder axis 300. In some embodiments, the first location 200A, where the first polymeric bead 103A is located, and the second location 200B, where the second polymeric bead 103B is located, can both be along the steep cylinder axis 302. For example, the first location 200A and the second location 200B can be at opposite ends of the steep cylinder axis 302.

The flat cylinder axis 300 can be measured by wavefront aberrometry. The steep cylinder axis 302 can be oriented substantially perpendicular or at right angles 304 (a 90-degree angle) with respect to the flat cylinder axis 300.

In certain embodiments, the flat cylinder axis 300 can be oriented at an oblique angle 306 (a non-90 degrees angle) with respect to a midline 308 substantially bisecting the optic portion 102.

For example, the oblique angle 306 can be a rotational angle with respect to the midline 308. As a more specific example, the oblique angle 306 can be about 30 degrees, about 45 degrees, about 60 degrees, about 120 degrees, about 135 degrees, about 150 degrees, or any degrees therebetween excluding 90 degrees.

As previously discussed, the intraocular lens 100 can comprise two haptics 104 coupled to the optic portion 102 via two haptic-optic interfaces 112. As shown in FIG. 3A, the midline 308 can substantially bisect the haptic-optic interfaces 112.

In certain embodiments, the optic portion 102 can also comprise a first pair of fluid channels 110A and a second pair of fluid channels 110B (see, e.g., FIG. 1A). The first pair of fluid channels 110A can be configured to place the fluid-filled optic portion 102 in fluid communication with the first haptic fluid lumen 106A and the second pair of fluid channels 110B can be configured to place the fluid-filled optic portion 102 in fluid communication with the second haptic fluid lumen 106B (see, e.g., FIG. 1A). The midline 308 can be oriented such that it extends in between or bisects the first pair of fluid channels 110A and the second pair of fluid channels 110B.

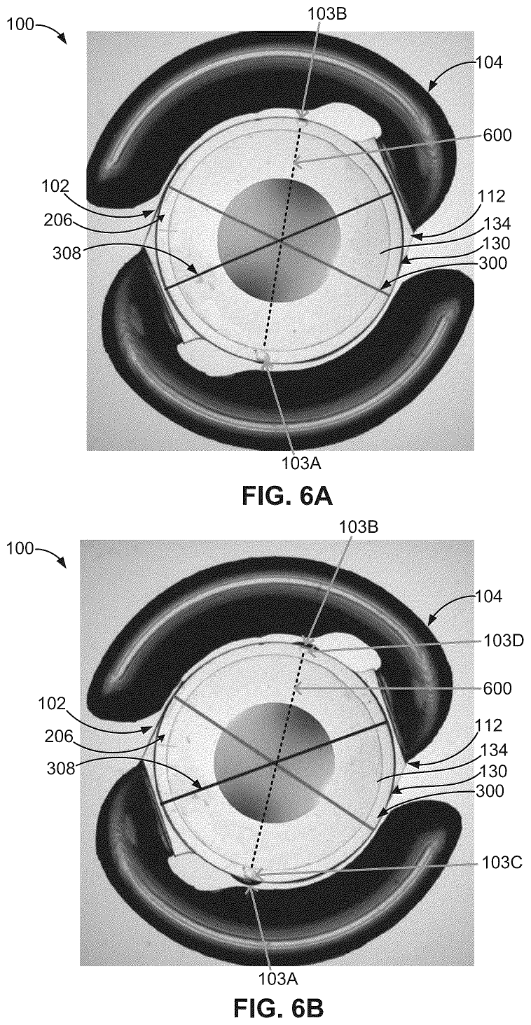

In alternative embodiments, the first polymeric bead 103A and the second polymeric bead 103B can be located along an axis that is oriented at an oblique angle (a non-90 degrees angle) with respect to the flat cylinder axis 300 (see, e.g., FIG. 6A and FIG. 6B). In these embodiments, the first location 200A and the second location 200B can be located along an axis that is oriented at an oblique angle (a non-90 degrees angle) with respect to the flat cylinder axis 300. Also, in these embodiments, the first polymeric bead 103A (and the first location 200A) and the second polymeric bead 103B (and the second location 200B) are not located along the steep cylinder axis 302 (see, e.g., FIG. 6A and FIG. 6B).

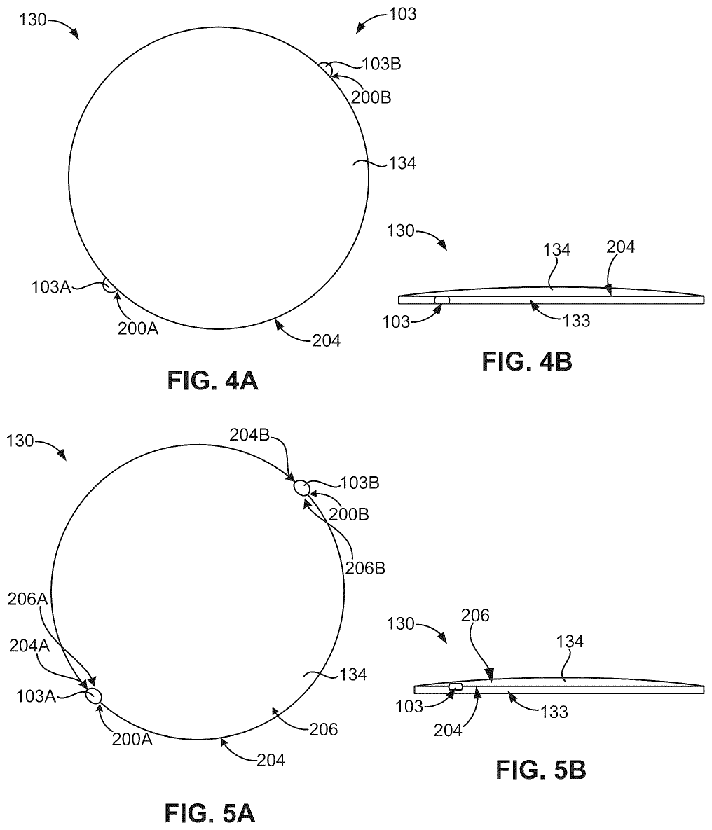

FIG. 4A is a top plan view of an anterior element 130 of the intraocular lens 100 with polymeric beads 103 applied to an exterior of the anterior element 130. FIG. 4B is a side view of the anterior element 130 of FIG. 4A.

FIGS. 4A and 4B illustrate that the anterior element 130 can have an anterior lateral surface 133 disposed along an exterior periphery of the anterior element 130. One or more polymeric beads 103 (e.g., the first polymeric bead 103A and the second polymeric bead 103B) can be located along or otherwise applied to the anterior lateral surface 133 of the anterior element 130.

As shown in FIG. 4A, the first polymeric bead 103A can be located at a first location 200A along the anterior lateral surface 133 and the second polymeric bead 103B can be located at a second location 200B along the anterior lateral surface 133. The first location 200A can be diametrically opposed to the second location 200B.

FIG. 5A is a top plan view of an anterior element 130 of the intraocular lens 100 with another type of polymeric beads 103 applied to an exterior of the anterior element 130. FIG. 5B is a side view of the anterior element 130 of FIG. 5A.

FIGS. 5A and 5B illustrate that the anterior outer surface 134 can meet the anterior lateral surface 133 at a circular edge 204 extending circumferentially around the anterior element 130. The anterior outer surface 134 can also comprise a rim portion 206 disposed around a periphery of the anterior outer surface 134 but radially inward of the circular edge 204.

The anterior outer surface 134 can further comprise a first rim portion 206A located immediately anterior to the first location 200A and a second rim portion 206B located immediately anterior to the second location 200B. The circular edge 204 can comprise a first edge location 204A and a second edge location 204B diametrically opposed to the first edge location 204A. The first edge location 204A can be located radially outward of the first rim portion 206A and the second edge location 204B can be located radially outward of the second rim portion 206B.

As shown in FIGS. 5A and 5B, at least part of the first polymeric bead 103A can be located or otherwise applied to the first location 200A and the remainder of the first polymeric bead 103A can be located or otherwise applied anterior to the first location 200A along the first rim portion 206A of the anterior outer surface 134. In this embodiment, the first polymeric bead 103A can cover the first edge location 204A.

Moreover, at least part of the second polymeric bead 103B can be located or otherwise applied to the second location 200B and the remainder of the second polymeric bead 103B can be located or otherwise applied anterior to the second location 200B along the second rim portion 206B of the anterior outer surface 134. In this embodiment, the second polymeric bead 103B can cover the second edge location 204B.

Although FIGS. 4A, 4B, 5A, and 5B illustrate the intraocular lens 100 comprising two polymeric beads 103, it is contemplated by this disclosure that the intraocular lens 100 can comprise three, four, or more (e.g., five, six, seven, eight, etc.) polymeric beads 103 (see, e.g., FIG. 6B).

FIG. 6A is a wavefront map of one embodiment of the intraocular lens 100 with polymeric beads 103 applied to an exterior of the intraocular lens 100. As previously discussed, the flat cylinder axis 300 of the intraocular lens 100 can be measured by wavefront aberrometry. For example, the flat cylinder axis 300 can be measured by a wavefront measurement system distributed by Optocraft GmbH.

FIG. 6A illustrates that the flat cylinder axis 300 can be oriented at an oblique angle with respect to a midline 308 substantially bisecting the intraocular lens 100. For example, the midline 308 can substantially bisect the haptic-optic interfaces 112 of the intraocular lens 100.

FIG. 6A also illustrates that the intraocular lens 100 can comprise a first polymeric bead 103A and a second polymeric bead 103B covering at least part of the rim portion 206 of the anterior outer surface 134 of the anterior element 130. The first polymeric bead 103A can be located or arranged diametrically opposed to the second polymeric bead 103B.

At least part of the first polymeric bead 103A and at least part of the second polymeric bead 103B can also be applied to or otherwise cover part of the anterior lateral surface 133 (see, e.g., FIGS. 1B, 1C, 2A, 2B, 3B, 4B, and 5B) of the anterior element 130.

As shown in FIG. 6A, the first polymeric bead 103A and the second polymeric bead 103B can be located along an axis 600 (e.g., at opposite ends of the axis 600) that is oriented at an oblique angle with respect to the flat cylinder axis 300. In this case, the axis 600 is not the steep cylinder axis 302 (see, e.g., FIG. 3A).

FIG. 6B is a wavefront map of another embodiment of the intraocular lens 100 with multiple polymeric beads 103 applied to an exterior of the intraocular lens 100. As shown in FIG. 6B, the polymeric beads 103 can comprise a first polymeric bead 103A, a second polymeric bead 103B, a third polymeric bead 103C, and a fourth polymeric bead 103D.

The first polymeric bead 103A and the second polymeric bead 103B can be located or otherwise applied to the anterior lateral surface 133 (see, e.g., FIGS. 1B, 1C, 2A, 2B, 3B, 4B, and 5B) of the anterior element 130. The first polymeric bead 103A can also be located or arranged diametrically opposed to the second polymeric bead 103B.

At least part of the third polymeric bead 103C can be located slightly anterior to the first polymeric bead 103A and cover part of the rim portion 206 of the anterior outer surface of the anterior element 130. Moreover, at least part of the fourth polymeric bead 103D can be located slightly anterior to the second polymeric bead 103B and cover another part of the rim portion 206 of the anterior outer surface 134 of the anterior element 130. The third polymeric bead 103C can be located or arranged diametrically opposed to the fourth polymeric bead 103D.

As shown in FIG. 6B, the first polymeric bead 103A, the second polymeric bead 103B, the third polymeric bead 103C, and the fourth polymeric bead 103D can be located along an axis 600 (for example, at opposite ends of the axis 600) that is oriented at an oblique angle with respect to the flat cylinder axis 300. In this case, the axis 600 is not the steep cylinder axis (see, e.g., FIG. 3A).

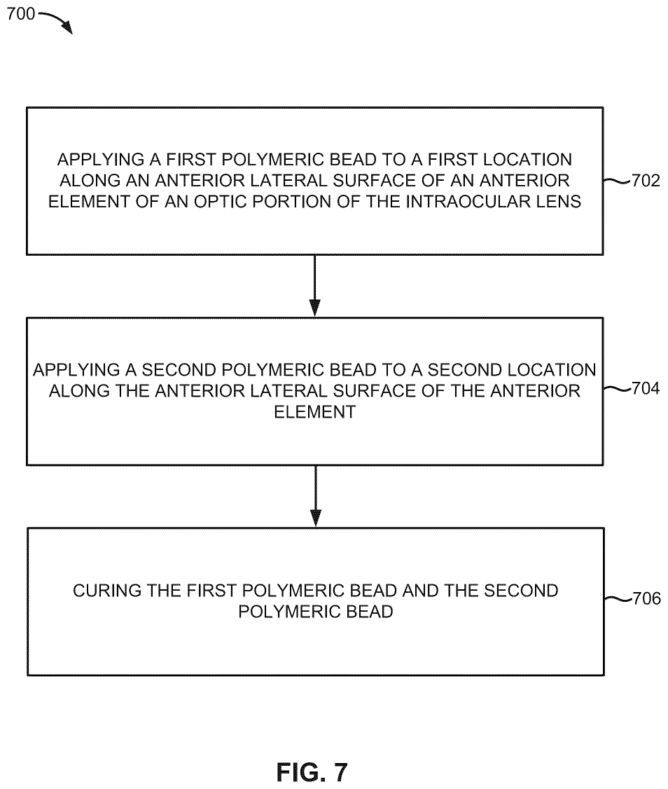

FIG. 7 is a flowchart illustrating a method 700 of reducing a cylinder power of an intraocular lens 100 (see, e.g., FIGS. 1A, 1B, 1C, 2A, 3A, 3B, 6A, and 6B). The method 700 can comprise applying a first polymeric bead 103A to a first location 200A along an anterior lateral surface 133 of an anterior element 130 of an optic portion 102 of the intraocular lens 100 (see, e.g., FIGS. 1A, 1B, 1C, 2A, 3A, 4A, 4B, 5A, 5B, 6A, and 6B) in step 702. The method 700 can also comprise applying a second polymeric bead 103B to a second location 200B along the anterior lateral surface 133 of the anterior element 130 (see, e.g., FIGS. 1A, 1B, 1C, 2A, 3A, 4A, 4B, 5A, 5B, 6A, and 6B) in step 704.

The anterior lateral surface 133 can be located along an exterior periphery of the anterior element 130. The first location 200A can be diametrically opposed to the second location 200B.

The method 700 can also comprise curing the first polymeric bead 103A applied at the first location 200A and curing the second polymeric bead 103B applied at the second location 200B in step 706.

In some embodiments, the first polymeric bead 103A and the second polymeric bead 103B can be cured by UV light. In certain embodiments, the first polymeric bead 103A and the second polymeric bead 103B can be cured between about 30 seconds and about 300 seconds.

The polymeric beads 103 can shrink between about 1% and 10% by volume after the curing step. When the polymeric beads 103 shrink, the polymeric beads 103 can add tension or induce certain tensile stresses on the optic portion 102 along an axis at which the polymeric beads 103 are placed.

In certain embodiments, the method 700 can also comprise measuring a cylinder power of the intraocular lens 100 after curing the first polymeric bead 103A and the second polymeric bead 103B. The method 700 can further comprise applying a third polymeric bead (e.g., the third polymeric bead 103C of FIG. 6B) and applying a fourth polymeric bead (e.g., the fourth polymeric bead 103D of FIG. 6B) to the anterior element 130 of the optic portion 102 of the intraocular lens 100. The third polymeric bead can be located diametrically opposed to the fourth polymeric bead.

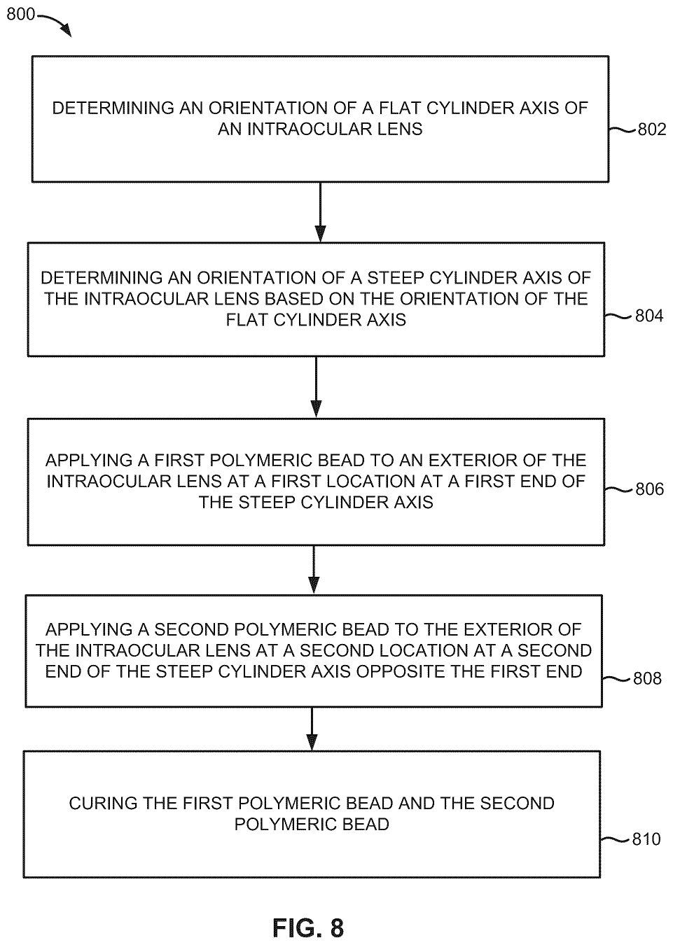

FIG. 8 is a flowchart illustrating a method 800 of adjusting a cylinder power of an intraocular lens 100 (see, e.g., FIGS. 1A, 1B, 1C, 2A, 3A, 3B, 6A, and 6B). The method 800 can comprise determining an orientation of a flat cylinder axis 300 (see, e.g., FIG. 3A) of the intraocular lens 100 in step 802. For example, the flat cylinder axis 300 can be determined using wavefront aberrometry. The method 800 can also comprise determining an orientation of a steep cylinder axis 302 (see, e.g., FIG. 3A) of the intraocular lens 100 based on the orientation of the flat cylinder axis 300 in step 804.

The method 800 can further comprise applying a first polymeric bead 103A to an exterior of the intraocular lens 100 at a first location 200A at a first end of the steep cylinder axis 302 (see, e.g., FIG. 3A) in step 806. The method 800 can also comprise applying a second polymeric bead 103B to the exterior of the intraocular lens 100 at a second location 200B at a second end of the steep cylinder axis 302 opposite the first end (see, e.g., FIG. 3A) in step 808.

The method 800 can also comprise curing the first polymeric bead 103A applied at the first location 200A and curing the second polymeric bead 103B applied at the second location 200B in step 810.

In certain embodiments, the method 800 can also comprise measuring a cylinder power of the intraocular lens 100 after curing the first polymeric bead 103A and the second polymeric bead 103B. The method 800 can further comprise applying a third polymeric bead (e.g., the third polymeric bead 103C of FIG. 6B) and applying a fourth polymeric bead (e.g., the fourth polymeric bead 103D of FIG. 6B) to the exterior of the intraocular lens 100. The third polymeric bead can be located diametrically opposed to the fourth polymeric bead.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications, and journal articles) are incorporated by reference herein in their entireties except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to +0.1%, +1%, +5%, or +10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An intraocular lens, comprising:
an optic portion, comprising:
an anterior element comprising an anterior lateral surface along an exterior periphery of the anterior element, and
a posterior element;
a first polymeric bead located at a first location along the anterior lateral surface; and
a second polymeric bead located at a second location along the anterior lateral surface, wherein the first location is diametrically opposed to the second location.

2. The intraocular lens of claim 1, wherein the optic portion has a flat cylinder axis and a steep cylinder axis substantially orthogonal to the flat cylinder axis.

3. The intraocular lens of claim 2, wherein the first location and the second location are located along the steep cylinder axis.

4. The intraocular lens of claim 2, wherein the first location and the second location are located along an axis that is oriented at an oblique angle with respect to the flat cylinder axis.

5. The intraocular lens of claim 2, wherein the flat cylinder axis is measurable by wavefront aberrometry.

6. The intraocular lens of claim 2, wherein the flat cylinder axis is oriented at an oblique angle with respect to a midline substantially bisecting the optic portion.

7. The intraocular lens of claim 1, wherein the anterior element further comprises an anterior outer surface disposed anterior to the anterior lateral surface, wherein at least part of the first polymeric bead is also located anterior to the first location along a first rim portion of the anterior outer surface, and wherein at least part of the second polymeric bead is also located anterior to the second location along a second rim portion of the anterior outer surface.

8. The intraocular lens of claim 7, wherein the anterior outer surface meets the anterior lateral surface at a circular edge, wherein the circular edge comprises a first edge location and a second edge location diametrically opposed to the first edge location, wherein the first polymeric bead covers the first edge location, and wherein the second polymeric bead covers the second edge location.

9. The intraocular lens of claim 1, wherein at least one of the first polymeric bead and the second polymeric bead has a bead volume of between about 0.0015 μL and about 0.05 μL.

10. The intraocular lens of claim 1, wherein at least one of the first polymeric bead and the second polymeric bead has a bead size comprising a bead diameter and a bead height, wherein the bead diameter is between about 0.2 mm and 0.5 mm, and wherein the bead height is between about 0.05 mm and 0.25 mm.

11. The intraocular lens of claim 1, wherein at least one of the first polymeric bead and the second polymeric bead is made of a formulation comprising one or more cross-linkable polymers and a reactive acrylic monomer diluent.

12. The intraocular lens of claim 1, wherein the first polymeric bead is applied to the anterior element and then cured by UV light, and wherein the second polymeric bead is applied to the anterior element and then cured by UV light.

13. The intraocular lens of claim 12, wherein curing the first polymeric bead to the anterior element and curing the second polymeric bead to the anterior element reduces a cylinder power of the optic portion by between about 0.1 diopters to 0.5 diopters.

14. The intraocular lens of claim 12, wherein curing the first polymeric bead to the anterior element and curing the second polymeric bead to the anterior element adjusts a cylinder power of the optic portion to a desired cylinder power.

15. The intraocular lens of claim 12, wherein curing the first polymeric bead to the anterior element and curing the second polymeric bead to the anterior element adjusts an angular location of a cylinder axis of the optic portion.

16. The intraocular lens of claim 1, wherein the intraocular lens is an accommodating intraocular lens.

17. The intraocular lens of claim 1, wherein the intraocular lens is a fluid-tunable non-accommodating intraocular lens.

18. A method of reducing a cylinder power of an intraocular lens, comprising:
applying a first polymeric bead to a first location along an anterior lateral surface of an anterior element of an optic portion of the intraocular lens, wherein the anterior lateral surface is located along an exterior periphery of the anterior element;
applying a second polymeric bead to a second location along the anterior lateral surface, wherein the first location is diametrically opposed to the second location; and
curing the first polymeric bead applied at the first location and curing the second polymeric bead applied at the second location.

19. The method of claim 18, wherein the optic portion has a flat cylinder axis and a steep cylinder axis substantially orthogonal to the flat cylinder axis.

20.-34. (canceled)

35. A method of adjusting a cylinder power of an intraocular lens, comprising:
determining an orientation of a steep cylinder axis of the intraocular lens;
applying a first polymeric bead to an exterior of the intraocular lens at a first location at a first end of the steep cylinder axis; and
applying a second polymeric bead to the exterior of the intraocular lens at a second location at a second end of the steep cylinder axis opposite the first end.

36.-51. (canceled)
